(19) United States
(12) Patent Application Publication
(10) Pub. No.: US 2009/0299267 A1
(43) Pub. Date: Dec. 3, 2009

(54) IONTOPHORETIC DRUG DELIVERY SYSTEM WITH PROCEDURE WINDOW

(75) Inventor: Emma Amelia DURAND, Jamestown, RI (US)

Correspondence Address: PILLSBURY WINTHROP SHAW PITTMAN, LLP, P.O. BOX 10500, MCLEAN, VA, 22102, US

(73) Assignee: Isis Biopolymer LLC, Warwick, RI (US)

(21) Appl. No.: 12/427,981

(22) Filed: Apr. 22, 2009

Related U.S. Application Data
(60) Provisional application No. 61/056,577, filed on May 28, 2008.

(51) Int. Cl. A61N 1/30

(52) U.S. Cl. 604/20

(57) ABSTRACT

The current invention is directed to an iontophoretic drug delivery system which drives charged drug molecules into a tissue, which comprises a body configured to be disposed on the tissue; a procedure window extending through said body, said window being open to provide access to the tissue for performance of a medical procedure on the tissue; a drug reservoir for holding the charged drug molecules; an electrode for driving the charged drug molecules into the tissue; and a controller configured to control the electrode.

### FIELD OF THE INVENTION

The present invention relates to the field of devices and systems for delivering drugs to medicate a patient, and more particularly to an iontophoretic drug delivery system.

The present application claims priority to U.S. Provisional Application Ser. No. 61/056,577, filed May 28, 2008, the entirety of which is incorporated herein by reference.

### BACKGROUND OF THE INVENTION

Iontophoresis is a drug delivery system. Iontophoresis is a non-invasive method of propelling charged molecules, normally medication or bioactive agents, transdermally by repulsive electromotive force. By applying a low level electrical current to a similarly charged drug solution, iontophoresis repels the drug ions through the skin to the underlying tissue. In contrast to passive transdermal patch drug delivery, iontophoresis is an active (electrically driven) method that allows the delivery of soluble ionic drugs through the skin.

An electrode drives charged molecules into the skin. Drug molecules with a positive charge are driven into the skin by an anode and those molecules with a negative charge are driven into the skin by a cathode.

There are a number of factors that influence iontophoretic transport including skin pH, drug concentration and characteristics, ionic competition, molecular size, current, voltage, time applied and skin resistance. Drugs typically permeate the skin via appendageal pores, including hair follicles and sweat glands.

Iontophoresis has numerous advantages over other drug delivery methods. The risk of infection is reduced because iontophoresis is non-invasive. Also, iontophoresis provides a relatively pain-free option for patients who are reluctant or unable to receive injections. For skin tissues, drug solutions may be delivered directly to the treatment site without the disadvantages of injections or orally administered drugs. Further, iontophoresis minimizes the potential for further tissue trauma that can occur with increased pressure from an injection.

### SUMMARY OF THE INVENTION

One aspect of the present invention provides an iontophoretic drug delivery system for driving charged drug molecules into a tissue. The system comprises a body configured to be disposed on the tissue with a procedure window extending through the body. The window is open to provide access to the tissue for performance of a medical procedure on the tissue. The system further comprises a drug reservoir for holding the charged drug molecules, an electrode for driving the charged drug molecules into the tissue; and a controller configured to control the electrode.

With the provision of the procedure window, a medical practitioner may perform a procedure through the window without having to remove the device. Without being limited to any specific use, it is envisioned that such a construction may be particular useful in dermatological procedures, such as biopsying or removing moles, warts, or other undesirable skin markings or other procedures such as cryotherapy, dermabrasion, etc. Likewise, the device may also be useful in emergency room or urgent care settings, such as for the focused delivery of a drug formulation to a specific site prior to or following performing a medical procedure on that site, including but not limited to anesthetics, antibiotics, anti-inflammatory drugs, etc.

The iontophoretic drug delivery system may contain different various numbers of drug reservoirs depending upon the particular treatment. Where a single drug is being delivered, the system may contain a single drug reservoir adjacent one electrode. Where a treatment requires two drugs that have oppositely charged solutions, the system may include a reservoir adjacent each of the oppositely charged electrodes. Where multiple drugs having the same charge are used, they may be either mixed into a single drug reservoir or placed in multiple drug reservoirs each adjacent a respective electrode having the same electric charge.

The size of the electrodes may vary in different embodiments depending upon the strength of the electrical current needed to be produced in order to drive drug molecules of various sizes into a patient's skin.

In one non-limiting embodiment, the electrodes and a microprocessor and battery are attached on opposite sides of a flexible sheet. The electrodes and the microprocessor and battery are electrically connected utilizing conductive silver ink. Through holes formed in the flexible sheet electrically connect the electrodes to the microprocessor and battery. The microprocessor and battery are attached to the system using conductive cement.

In another embodiment, the system may contain various sensors to measure parameters such as patient skin temperature, moisture at the system/patient skin interface, or other patient or drug delivery related parameters.

Other objects, features and aspects of the invention will become apparent from the following detailed description, the accompanying drawings, and the appended claims.

### BRIEF DESCRIPTION OF THE DRAWINGS

The novel features that are considered characteristic of the invention are set forth with particularity in the appended claims. The invention itself; however, both as to its structure and operation together with the additional objects and advantages thereof are best understood through the following description of the preferred embodiment of the present invention when read in conjunction with the accompanying drawings, wherein:

FIG. 1 shows a front view of an iontophoretic drug delivery system 10; and

FIG. 2 is a rear view of the device of FIG. 1.

### DETAILED DESCRIPTION OF THE ILLUSTRATED EMBODIMENT(S)

While the invention has been shown and described with reference to a particular embodiment thereof, it will be understood to those skilled in the art, that various changes in form and details may be made therein without departing from the spirit and scope of the invention.

Reference may be made to commonly owned U.S. Provisional Applns. 61/012,582, filed Dec. 10, 2007 and 60/956,558, filed Aug. 17, 2007, for examples of iontophoretic devices and a description of their operation. Reference may also be made to commonly owned U.S. patent application Ser. No. 12/330,698 filed Dec. 9, 2008, and Ser. No. 12/192,540 filed Aug. 15, 2008. These applications are incorporated herein by reference in their entirety, and the present invention may be applied to those devices.

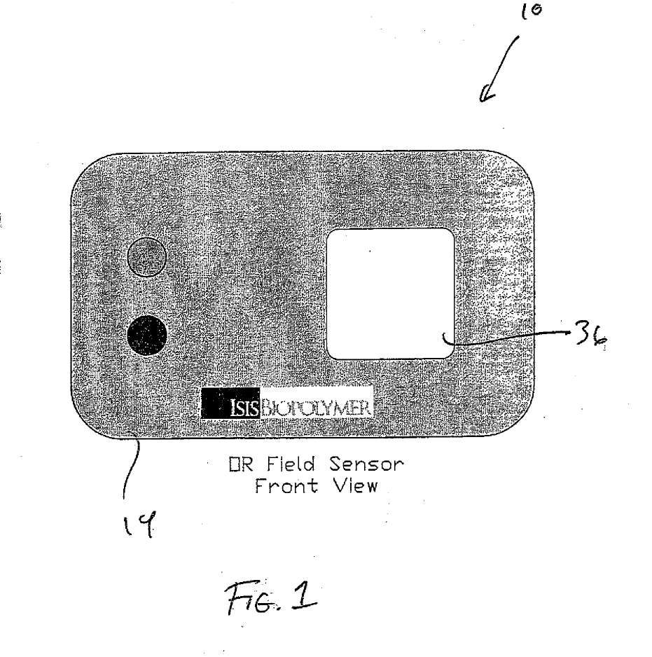

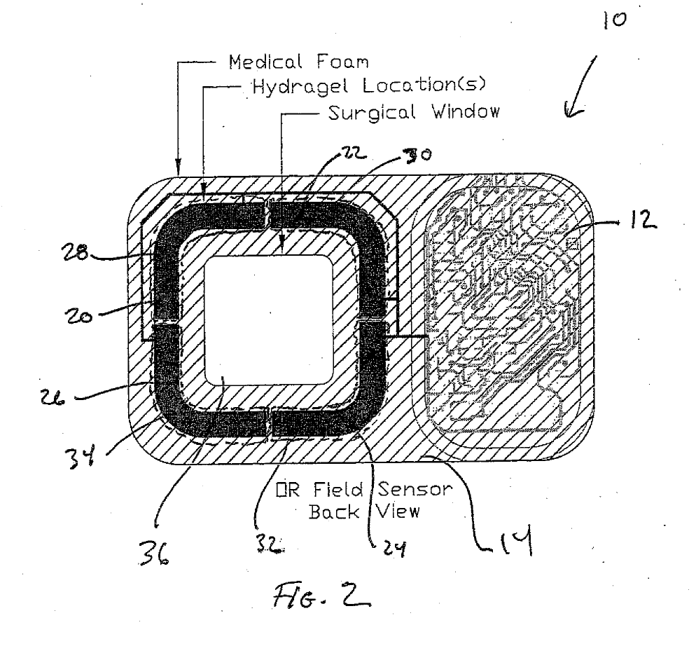

FIG. 1 is a front view of an iontophoretic drug delivery system 10, and FIG. 2 is a rear view of the same. The rear side is the side that faces the patient's tissue during treatment, and the front side is the side that faces away. System 10 provides a non-invasive method of propelling high concentrations of a charged substance, normally medication or bioactive-agents, transdermally by repulsive electromotive force. The iontophoretic drug delivery system 10 includes control and power supply circuitry shown generally and schematically at 12 and which may include a microprocessor controller, a battery, and printed flexible wiring on the main body 14. The construction and operation of the circuitry is not essential, and it may take any form. For further details, reference may be made to either of the two above-incorporated applications for details in this regard.

The illustrated embodiment includes four electrodes 20, 22, 24 and 26 mounted to the main body 14. Each of these electrodes has an associated drug reservoir 28, 30, 32, and 34 (shown in dashed lines) adjacent to it. The construction for providing the electrodes and the drug reservoirs is the same as described in the two above-incorporated applications. Here, however, the shape and location of the electrodes are modified to accommodate the procedure window 36, discussed below in further detail.

The use of an optional antenna provides a wireless capability for system 10 to communicate with other external devices. In an exemplary embodiment, the antenna may be an RFID antenna, a blue-tooth enabled device, an infra-red wireless device, or another wireless signal receiver. The antenna may function as an RFID antenna or can receive signals from an outside device through capacitive coupling. The antenna can also be configured in the shape of inductive coils in order to receive signals from an outside device through inductive coupling. Further details regarding the antenna are described in the above-incorporated applications.

As is also described in the two above-incorporated applications, adhesive is applied to the rear face (i.e., the tissue contacting surface) of the body 14. As an option, the adhesive may comprise two adhesives of different tacks as described in those incorporated applications. A low-tack adhesive may placed within the internal area of the tissue contacting surface of body 14, and a high-tack adhesive may extend around the periphery of body 14 and secure the outer edge of system 10 to the skin of a patient. A preferred type of adhesive for the high-tack adhesive is a silicone-based adhesive that is rapidly cured with an electron beam or UV radiation. Preferably, the adhesive is not present between the drug reservoirs and the skin, as this contact could alter the properties of adhesive and/or influence the release of the drug. Where the drug reservoir gel is tacky, a higher tack adhesive may be used along the outer-peripheral edge to provide a stable bond to the skin to discourage removal or de-lamination from the skin. However, the drug reservoir may comprise a drug soluble in a gel, such as a hydrogel, and the gel may be tacky so as to promote direct bonding to the patient's skin (i.e., without;the use of an intervening adhesive between the reservoir and the skin).

Prior to use, a release layer (not shown) is placed over adhesive for protection. The release layer is removed from system 10 just prior to bonding system 10 to the skin of a patient. The release layer makes sufficient contact with the adhesive to hold it to system 10 while allowing a user to easily peel the release layer off of system 10. Typically, the release layer is coated with a silicone based release coating to ensure that it can be peeled off without degrading the adhesives.

The procedure window 36 is provided and is configured to enable a medical practitioner to perform a procedure through the window without having to remove the device. Without being limited to any specific use, it is envisioned that such a construction may be particular useful in dermatological procedures, such as biopsying or removing moles, warts, or other undesirable skin markings or other procedures such as cryotherapy, dermabrasion, etc. Likewise, the device may also be useful in emergency room or urgent care settings, such as focused delivery of a drug formulation to a specific site prior to or following performing a medical procedure on that site, including but not limited to anesthetics, anti-inflammatory drugs, etc.

One advantage of the device is that it delivers the drug molecules without penetrating the skin (as is the case with a needle). This enables the device to be used to deliver a local anesthetic, such as lidocaine, to the tissue in advance of the procedure without using a needle. Because a needle penetrates the skin, it is usually advisable to have the patient in a sterile area (e.g., a patient treatment room) for the injection, and also for any treatment to the site to follow. However, the patient will typically remain in the sterile area while waiting for the anesthetic to take effect, which means that the area cannot be used for treating another patient in the meantime. With the present invention, because no needle is needed, the device may be used to deliver the local anesthetic while the patient is waiting in a non-sterile area (such as a waiting room), thus necessitating less waiting time in the patient area. This may have particular applicability in dermatologists offices, emergency rooms, urgent care clinics, etc.

Also, because the device 10 can remain on the user before and after the procedure (as the procedure is performed through the window 36 without removing the device), the device can be used to deliver medications that may be useful in the post-procedure context. For example, the device 10 may continue to deliver local anesthetic to relieve post-procedure pain/discomfort or it may include an anti-inflammatory medication in one or more of its drug reservoirs. Or an anti-biotic medication may be included in one or more of the drug reservoirs. As such, the device can be used a post-procedure tool delivering such drugs to the treatment site. As an option, appropriate logic may be included in the processor to select between delivery of various substances, such as may be triggered based on time, or upon the medical professional using a switch to indicate completion of the procedure.

In a non-limiting embodiment, the device may include one or more reservoirs with a local anesthetic, and one or more reservoirs with a post-treatment medication. For example, drug reservoirs 28 and 32 may include a local anesthetic, and drug reservoirs 30 and 34 may include an anti-inflammatory medication, such as an analgesic. Thus, during use the device may initially be operated to deliver the local anesthetic to the tissue from drug reservoirs 28 and 32 prior to the medical procedure being performed through window 36. And then after completion of the procedure, the device may be set (e.g., by operation of a switch) to deliver the anti-inflammatory medication from drug reservoirs 30 and 34. Likewise, the device could deliver both medications before and after the procedure. Any protocol for drug delivery may be used, and this example is not intended to be limiting.

As described in the above-incorporated applications, charged drug molecules are contained within the reservoirs 28, 30, 32, and 34, which face the patient's skin through openings. The drug reservoirs may each be a gel pad or membrane to which the charged drug molecules contained in a solution are applied or injected. By impregnating a gel pad or membrane with charged drug molecules, the charged drug molecules are not able to readily be absorbed into a patient's body without the operation of electrodes. In one embodiment, drug reservoirs are a conductive medium to support the function of electrodes. By making drug reservoirs also a conductive medium, system 10 can function with a lower amount of current, thereby extending battery life and reducing the amount of current put into a patient's skin, of which a high amount of current can cause irritation. Typically, the solution is injected through a port into drug reservoirs. Electrodes drive the charged drug molecules out of drug reservoirs into the skin of a patient. Where the reservoir includes a gel, the drug in ionic form may be mixed with the gel matrix cured together and assembled into the system 10.

The basis of ion transfer lies in the principle that like poles repels and unlike poles attract. Ions, being particles with a positive or a negative charge are repelled into the skin by an identical charge the electrode places over it. When a direct electric current activates electrodes, anions in the solution, ions with a negative charge, are repelled from the negatively charged electrode. Positively charged ions (cations) are likewise repelled from the positive electrode. The electrical current drives ions through the skin that would not be absorbed passively. The quantity of ions that are made to cross the skin barrier is proportional to the current density and to the amount of time the current flows through the solution. Current density is determined by the strength of electric field and the electrode size. A desired current strength is in the range of 0.4 mA or 2.0 mA per square inch of electrode surface. This current strength is below sensory perception of a typical human patient. If electrodes are too small, thereby concentrating the current (or if the current is too high), it may be more uncomfortable for the patient, as the current density may be sensed as an irritant.

In the illustrated embodiment, the electrodes and drug reservoirs preferably extend at least partially about the periphery of window 36, as that is the site where treatment is targeted. As shown in the Figures, the electrodes more preferably extend about the entire periphery of the window 36.

As is discussed in the above-incorporated applications, the electrodes and flexible printed wiring are preferably (but optionally) made from a flexible material that can bend with layer in conformity to the application area of the patient's body. That is, a flexible and conformable base material is used, and the electrodes and wiring/circuitry are printed from a flexible material, thus enabling the whole device 10 to be flexed/bent to conform to the patient's body. One exemplary flexible material is silver conductive ink with resistivity of 8 to 10 milliohms per square. The resistivity of silver conductive ink within the range of 8 to 10 milliohms per square is desirable in order to have sufficient current to drive drugs into the stratum corneum. The ink may be silver (Ag), for example, and may be printed (e.g. by screen printing or gravure rolling) onto the layer. Most commercially available silver conductive inks have a resistivity in the range of 14 to 18 milliohms per square, which limits the current available to drive the drugs through the stratum corneum. The electrodes may be formed of silver chloride (AgCl).

Preferably, the printed ink is deposited by as polymer thick film circuitry, which is performed by mixing fine conductive particulates (e.g., silver or silver chloride) with polymer particulates, and then printing and curing that mixture to form the wiring/electrode(s). This impacts a high degree of flexibility because of the polymer constituent of the circuitry/electrodes, while the conductive constituent provides conductivity.

In many treatments, a single drug is used. However, it is common for the efficacy of many drugs to be increased by combining their delivery with other drugs. Thus, system 10 may be configured to deliver multiple types of charged drug molecules. In the case where the multiple drug molecules have the same charge, those drugs may be combined into a single solution and delivered from a single drug reservoir. In other embodiments where the multiple drugs have the same charge, but need to be delivered to the patient at different times or in different quantities, the multiple electrodes with multiple drug reservoirs may be used. In a case where there are two drugs having molecules of opposite polarity, different electrodes are provided with drug reservoirs for delivering their respective drugs to the patient. In one embodiment, drug reservoirs are formed of hydro-gel (i.e., a water-based gel). In another embodiment, drug reservoirs are formed on a membrane. The size of the electrodes will vary depending upon the size of the charged drug molecule that they are trying to repel into the patient's skin. Thus, in embodiments where multiple electrodes with multiple drug chambers are used, the sizes of the electrodes and drug chambers may vary.

One or more of the electrodes may be made of Ag/AgCl printable conductive ink coating, as described in the above-incorporated applications. The electrodes may be printed to the flexible printed wiring with a highly conductive Polymer Thick Film (PTF) ink. In a preferred embodiment, a lead-free, silver loaded isotropic conductive cement is used that provides an electrical and mechanical connection having resistance to moisture and thermal shock.

A battery powers the system 10. It is desirable to make the battery as thin as possible, along with the rest of system 10, in order to enhance the ability of system 10 to adhere to a patient's skin with minimal disruption to the patient. Battery cells on the order of 0.7 mm thickness can generate up to 3.0 volts of electricity and multiple arrays can generate and control up to 9.0 volts of electricity. This amount of power allows for wireless programming and data acquisition with a microprocessor controller through an antenna. The type and construction of the battery is not intended to be limiting. Reference may be made to the above-incorporated applications for further details in this regard.

The rate, timing and pattern of drug delivery using iontophoretic drug delivery system 10 are controlled by varying the electrical current applied to the electrodes. Reference may also be made to the above-incorporated applications for further teachings in this regard.

As an option, the electrodes, flexible printed wiring, antenna and other circuitry components in system 10, in a preferred embodiment, are made from Polymer Thick Film (PTF) flexible circuits that are manufactured using a technology that consists of a low-cost polyester dielectric substrate and screen-printed thick film conductive inks. These circuits are made with an additive process involving the high-speed screen printing of conductive ink. Multi-layer circuits are manufactured using dielectric materials as an insulating layer, and double-sided circuits using printed through-hole technologies. Further details on the optional use of this technology are provided in the above-incorporated applications. It is advantageous to utilize PTF flexible circuits because they are inherently less costly than, for example, copper based circuits. PTF are formed on a flexible dielectric substrate that circuit traces are printed directly upon. In addition, PTF typically uses a PET substrate which is significantly less expensive than the polyimide substrate which is commonly used in copper circuitry. In addition, as PTF circuits are more environmentally friendly as they are printed directly and do not require the removal of materials where chemicals are used to selectively etch away the copper foil to leave behind a conductive pattern.

The charged drug molecules vary in size for different drug compounds. Larger drug molecules require stronger electromagnetic forces to drive them into the skin of a patient. Smaller drug molecules require lesser electromagnetic forces to drive them into the skin of a patient. Thus, it is desirable to vary the size of electrodes the based upon the size of the drug compounds in order to deliver an optimal amount of electromagnetic force to drive the drug molecules into the patient's skin. System 10 is therefore preferably manufactured for a specific drug molecule size by having a tailored size for each electrode

The table shown below provides an exemplary list of drugs, the charge of the drug molecules and solution, and the purpose/condition for which the drugs are used.

| | Charge of | |
|---|---|---|
| | Solution/Drug | |
| Drug | Molecules | Purpose/Condition |
| Acetic acid | − | Calcium deposits |
| Atropine sulphate | + | Hyperhidrosis |
| Calcium | + | Myopathy, myospasm |
| Chloride | − | Sclerolytic, scar tissue |
| Citrate | − | Rheumatoid arthritis |
| Copper | + | Astringent |
| Dexamethasone | − | Tendinitis, bursitis |
| Glycopyrronium bromide | + | Hyperhidrosis |
| Iodine | − | Sclerolytic, scar tissue |
| Lidocaine | + | Dermal anesthesia |
| Magnesium | + | Muscle relaxant |
| Penicillin | − | Infected burn wounds |
| Poldine methyl sulfate | − | Hyperhidrosis |
| Potassium iodide | − | Scar Tissue |
| Salicylate | − | Analgesic, plantar warts |
| Sodium chloride | − | Scar tissue |
| Silver | + | Chronic osteomyelitis |
| Zinc | + | Antiseptic, wound healing |

In various embodiments, the flux of charged drug molecules from drug reservoirs into the patient's skin can be increased through the use of a skin permeation enhancer. A permeation enhancer is any chemical or compound that, when used in conjunction with the charged drug molecule, increases the flux of charged drug molecules from drug reservoir into the skin of the patient. That is, skin permeation enhancers are a substance that enhances the ability of the charged drug molecule transfer from the drug reservoir and permeate into the patient's skin.

Such use of a permeation enhancers is advantageous because it reduces the amount of electrical power required to transfer the drug from a reservoir and into the patient's skin. This means that less current can be used, which in turn reduces the potential for skin irritation. And it also means less power is drawn, meaning the battery can be made smaller and/or last longer.

The permeation enhancer need not be in the reservoir with the drug, and could be applied to the skin contacting surface of the reservoir. This could help create an interface between the reservoirs and the skin for enhancing permeation of the drug. Further details on the permeation enhancer are available in the above incorporated applications.

As an optional feature, the device 10 may be provided with a cover flap (not shown). The cover flap may be movable between an open position uncovering the procedure window 36 and a closed position covering the procedure window 36. This allows the medical practioner to open the window for performing the procedure, and to close it afterwards to cover the treatment site.

While the invention has been shown and described with reference to a particular embodiment thereof, it will be understood to those skilled in the art, that various changes in form and details may be made therein without departing from the spirit and scope of the invention.

What is claimed:

1. An iontophoretic drug delivery system driving charged drug molecules into a tissue, comprising:
a body configured to be disposed on the tissue;
a procedure window extending through said body, said window being open to provide access to the tissue for performance of a medical procedure on the tissue;
a drug reservoir for holding the charged drug molecules;
an electrode for driving the charged drug molecules into the tissue; and
a controller configured to control the electrode.

2. A system according to claim 1, wherein said drug reservoir extends about a periphery of said window.

3. A system according to claim 2, wherein said drug reservoir extends about the entire periphery of said window.

4. A system according to claim 1, wherein the charged drug molecules are charged molecules of an anesthetic.

5. A system according to claim 1, wherein the charged drug molecules are charged molecules of an antibiotic.

6. A system according to claim 1, wherein the charged drug molecules are charged molecules of an anti-inflammatory medication.

7. A system according to claim 1, further comprising a cover flap movable between an open position uncovering said procedure window and a closed position covering said procedure window.
